(19) United States
(12) Patent Application Publication
(10) Pub. No.: US 2008/0103402 A1
(43) Pub. Date: May 1, 2008

(54) Motion detection system for an external defibrillator

(75) Inventors: Ronald E. Stickney, Edmonds, WA (US); Daniel W. Piraino, Seattle, WA (US); Patricia J. O'hearn, Mercer Island, WA (US); Cynthia P. Jayne, Redmond, WA (US)

Correspondence Address: MARY Y. REDMAN;MEDTRONIC, INC., 710 MEDTRONIC PARKWAY NE M.S. LC340, MINNEAPOLIS, MN, 55432-5604, US

(73) Assignee: Medtronic Emergency Response Systems, Inc.

(21) Appl. No.: 11/590,610

(22) Filed: Oct. 30, 2006

(51) Int. Cl. A61B 5/04

(52) U.S. Cl. 600/509

(57) ABSTRACT

A medical device capable of analyzing a patient's ECG, such as an external defibrillator, may be operated according to a method including the steps of: (a) determining if the patient is undergoing motion; (b) if it is determined that the patient is undergoing motion, providing an indication of patient motion; (c) after providing the indication, determining if the patient is undergoing motion, and if the patient is undergoing motion, starting ECG analysis after an elapsed time T₀. The elapsed time T₀ may be measured from a point in time prior to the commencement of step (a). The method may further include the steps of collecting ECG data of the patient, and initiating analysis of the ECG data prior to step (b). The step (b) may further include the step of interrupting the prior initiated ECG analysis. The method may further include the step of starting a timer when the ECG analysis is commenced, to measure elapsed time.

### BACKGROUND OF THE INVENTION

1. Field of the Invention

The present invention relates to the field of external, implantable, or subcutaneous defibrillators, and in particular to motion detection systems for external, implantable, or subcutaneous defibrillators.

2. Technical Background

Sudden cardiac arrest (SCA) is a life-threatening cardiac emergency, typically caused by ventricular fibrillation, a condition where the human heart is unable to pump the volume of blood required by the human body. The generally accepted therapy for restoring a normal rhythm to a heart experiencing ventricular fibrillation (VF) is to apply electrical defibrillation therapy. This is the application of a strong electric pulse to the heart, which can be done using an external, implantable, or subcutaneous cardiac defibrillator. In recent years, automated external defibrillators, known as AEDs, have been successfully used by emergency responders such as firefighters, police officers and laypersons, having minimal or no medical training. AEDs are now easily accessible in many public places for use by lay rescuers while waiting for professional emergency medical service providers to arrive on the scene.

Another important therapy in a successful response to a cardiac emergency is cardiopulmonary resuscitation, commonly referred to as CPR. CPR includes a technique in which the patient's chest is rhythmically compressed in order to compress the heart so as to force blood flow through the circulatory system, and ventilation of the patient by mouth-to-mouth breathing, with or without a intervening mask, or using a bag valve mask assembly where the rescuer squeezes a bag which forces air into a mask placed around the patient's mouth and nose. Although some professional emergency medical responders use automated mechanical chest compression devices to administer chest compressions, chest compressions are more commonly applied to a patient manually, with the rescuer's hands on the patient's chest, and the rescuer's body providing the compressive force.

Commercially available AEDs analyze the patient's ECG to determine whether the heart is in a condition where a defibrillating shock is appropriate therapy.

In the treatment of SCA, to be successful, treatment by defibrillating shock must be given within a very few minutes. Thus, every second counts.

In the SCA case where treatment by defibrillating shock is not needed, the appropriate therapy may include CPR. There is a growing recognition that in SCA the amount of time spent performing CPR can influence the patient's chance for survival. Every second counts whether the appropriate therapy at the time is a defibrillating shock or CPR.

Excessive patient motion (for example, from chest compressions during CPR, vehicle movement, or movement of the patient on a stretcher) has long been known to adversely affect the reliability of detected ECG data and its analysis. Conventional AEDs have algorithms for detecting excessive motion (for example, motion due to CPR) and interrupting ECG analysis if excessive motion is detected. ECG analysis may not be restarted until a cessation of excessive motion is detected. In some cases, there may be considerable delay in restarting ECG analysis, or the device may prompt for another cycle of CPR if excessive motion is ongoing, which will delay ECG analysis for the duration of the CPR cycle. But, for treatment of VF to be successful, a defibrillating shock must be given within a very few minutes of its onset. Thus, every second counts. Some AEDs allow for motion detection to be turned off, out of concerns for delays in ECG analysis caused by motion detection.

### SUMMARY

In a first aspect, a method of operating a medical device capable of analyzing a patient's ECG includes the steps of: (a) determining if the patient is undergoing motion; (b) if it is determined that the patient is undergoing motion, providing an indication of patient motion; (c) after providing the indication, determining if the patient is undergoing motion, and if the patient is undergoing motion, starting ECG analysis after an elapsed time T₀. The elapsed time T₀ may be measured from a point in time prior to the commencement of step (a).

The method may further include the steps of collecting ECG data of the patient, and initiating analysis of the ECG data prior to step (b). The step (b) may further include the step of interrupting the prior initiated ECG analysis. The method may further include the step of starting a timer when the ECG analysis is commenced, to measure elapsed time.

In an embodiment, T₀ may be substantially equal to or less than 30 seconds, or it may be substantially equal to or less than 10 seconds.

In the method, determining if the patient is undergoing motion may include sensing patient impedance and analyzing the sensed patient impedance.

The step of starting the ECG analysis after an elapsed time T₀ may include the step of continuing the ECG analysis that had been interrupted in step (b). The step of starting the ECG analysis after an elapsed time T₀ may include the step of commencing a new ECG analysis.

The indication may include a prompt informing the user that motion is present. The prompt may include an instruction to the user to stop motion. The prompt may include an instruction to the user to not touch the patient.

In another aspect, a defibrillator includes electrodes attachable to a patient and configured to sense ECG signals and impedance, and a processor capable of analyzing the sensed impedance to detect if motion is present; and, if motion is present, waiting for an elapsed time T₀ before analyzing the ECG signals.

The defibrillator may further include a user interface controlled by the processor, wherein the processor causes the user interface to inform the user if the processor determines that motion is present. The user interface may provide a prompt to the user to stop motion if the processor determines that motion is present. The prompt may include a voice prompt instructing the user to not touch the patient.

In another aspect, a method of operating a defibrillator having electrodes attached to a patient includes the steps of: starting an ECG analysis of the patient; starting a measurement of elapsed time when the ECG analysis is started; analyzing a patient parameter to determine if the patient is undergoing motion which would influence ECG analysis; and if the analyzing step determines that the patient is undergoing motion which would influence ECG analysis, and the time measurement shows that an elapsed time of T₀ has not been reaches, interrupting the ECG analysis. This method may further include the step of: after a time T₀ has been reached, analyzing the ECG. The patient parameter may be impedance.

In this method, the step of analyzing the ECG after a time T₀ has been reached may includes the step of continuing the ECG analysis which had been interrupted. The step of analyzing the ECG after a time T₀ has been reached may include the step of commencing a new ECG analysis.

In some embodiments, the method may further include the step of: if the analyzing step determines that the patient is undergoing motion which would influence ECG analysis and the time measurement shows that an elapsed time of T₀ has not been reached, analyzing a second data set of a patient parameter to determine if the patient is undergoing motion which would influence ECG analysis; and if motion which would influence ECG analysis is present, waiting for an elapsed time T₀ before analyzing the ECG signals.

In some embodiments, T₀ may be substantially equal to 30 seconds, or it may be substantially equal to 10 seconds. The time T₀ may be a time that is chosen prior to application of the electrodes to the patient.

### BRIEF DESCRIPTION OF THE DRAWINGS

FIG. 1 is a block diagram of an external defibrillator; and

FIG. 2 is a flowchart illustrating a process according to an embodiment of the invention.

### DETAILED DESCRIPTION

Reference will now be made in detail to an embodiment of the invention, an example of which is illustrated in the accompanying drawings. Wherever possible, the same reference numbers are used throughout the drawings to refer to the same or like parts.

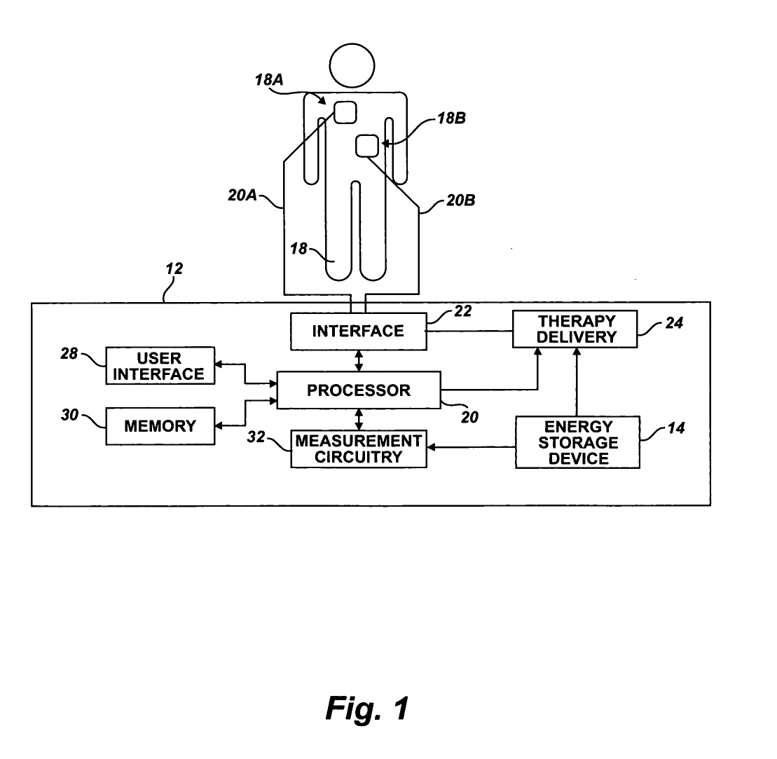

Referring to FIG. 1, an illustrative embodiment may be used in an external defibrillator 12. The circuitry in the defibrillator 12 may be substantially like that of commercially available external defibrillators such as the LIFEPAK® CR Plus defibrillator or the LIFEPAK 500 defibrillator sold by Medtronic Emergency Response Systems Inc. of Redmond, Wash. Defibrillator 12 administers defibrillation therapy to patient 10 via electrodes 14 and 16, which may be adhesive electrode pads placed on the skin of patient 10. The body of patient 10 provides an electrical path between electrodes 14 and 16.

Electrodes 14 and 16 are coupled to defibrillator 12 via conductors 18 and 20 and interface 22. Electrical impulses or signals may be sensed by defibrillator 12 via electrodes 14 and 16 and interface 22 for analysis by the microprocessor 26 to ascertain if the heart is in a condition which requires the application of defibrillation therapy (this is referred to as ECG analysis below). Electrical impulses or signals may also be delivered from defibrillator 12 to patient 10 via electrodes 14, 16, and interface 22. Interface 22 includes a switch (not shown in FIG. 1) that, when activated, couples an energy storage device 24 to electrodes 14 and 16. Energy storage device 24 stores the energy for a dosage of electrical energy or current to be delivered to patient 10.

Energy storage device 24 includes components, such as one or more capacitors, which store the energy to be delivered to patient 10 via electrodes 14 and 16. Before a defibrillation pulse may be delivered to patient 10, energy storage device 24 must be charged. The microprocessor 26 directs a charging circuit 28 to charge energy storage device 24 to a high voltage level. When the ECG analysis determines that defibrillation therapy is indicated, microprocessor 26 will automatically direct charging circuit 28 to begin charging. Or, in an alternative embodiment, microprocessor 26 will direct charging circuit 28 to begin charging upon the instruction of the rescuer. The rescuer may instruct microprocessor with one or more input devices 30A-30N (hereinafter 30), such as one or more buttons, a keyboard, a touch screen, a voice recognition module or a pointing tool. Microprocessor 26 monitors and analyses electrocardiogram (ECG) signals sensed via electrodes 14 and 16 and received via interface 22 and, in some embodiments, may display these signals via an output device 34 such as a display screen.

In the illustrated embodiment, when the analysis of the ECG indicates that defibrillation therapy is required, the microprocessor 26 starts the capacitor charging process. Capacitor charging circuit 28 includes, for example, a flyback charger that transfers energy from a power source 32 to energy storage device 24. Power source 32 may include one or more replaceable battery and/or an adapter to an exterior power source such as an electrical outlet. In addition to supplying energy to charging circuit 28 and energy storage device 24, power source 32 also supplies power to components such as microprocessor 26, input devices 30 and output devices 34, e.g., via a power supply circuit (not shown in FIG. 1). When the energy stored in energy storage device 24 reaches the desired level, defibrillator 12 is ready to deliver the defibrillation shock.

In one embodiment the shock may be delivered automatically without further input by the rescuer. In this case, defibrillator 12 notifies the rescuer that charging is complete using one or more output devices 34A-34N (hereinafter 34), such as a display screen, an audible sound generator, a voice synthesizer, or an indicator light, and instructs the rescuer to stand clear and not touch the patient 10. The defibrillator 12 then delivers the defibrillating shock to the patient. In another embodiment, the microprocessor 26 may activate an output device 34 that informs the rescuer that defibrillator 12 is ready to deliver a defibrillation shock to patient 10 and instructs the rescuer to activate the switch by manually operating an input device 30, such as by pressing a “shock” button. Defibrillator 12 then delivers a defibrillation shock to patient 10.

In addition to controlling the delivery of a defibrillation pulse, microprocessor 26 may also modulate the electrical pulse delivered to patient 10. Microprocessor 26 may, for example, regulate the shape of the waveform of the electrical pulse and the duration of the pulse. In addition, microprocessor 26 may evaluate the efficacy of an administered defibrillation shock. Furthermore, microprocessor 26 may store and retrieve data from memory 36. Memory 36 stores instructions that direct the operation of microprocessor 26. In addition, memory 36 stores information about patient 10 and defibrillator 12. For example, memory 36 may store the ECG of patient 10, information about the number of shocks delivered to patient 10, the energy delivered per shock, the timing of shocks and the patient response to shocks.

Examples of motion detection systems are found in commercially available AEDs such as those available under the trademarks LIFEPAK 500 and LIFEPAK CR Plus from Medtronic Emergency Response Systems. Inc. of Redmond Wash. Examples of motion detection systems are described in detail in U.S. Pat. Nos. 5,247,939 and 4,619,265, both of which are hereby incorporated by reference herein. In an example of a motion detection system, patient impedance data are collected and successive impedance values are compared to a predetermined threshold. If the last two values exceeded the threshold, then excessive motion is determined to be present. In another example, patient impedance values are compared to predetermined upper and lower limits, and the time in which they exceed these limits is noted. Excessive motion is determined to be present if the impedance signal undergoes relatively large variations for a short time, or smaller variations for a longer time.

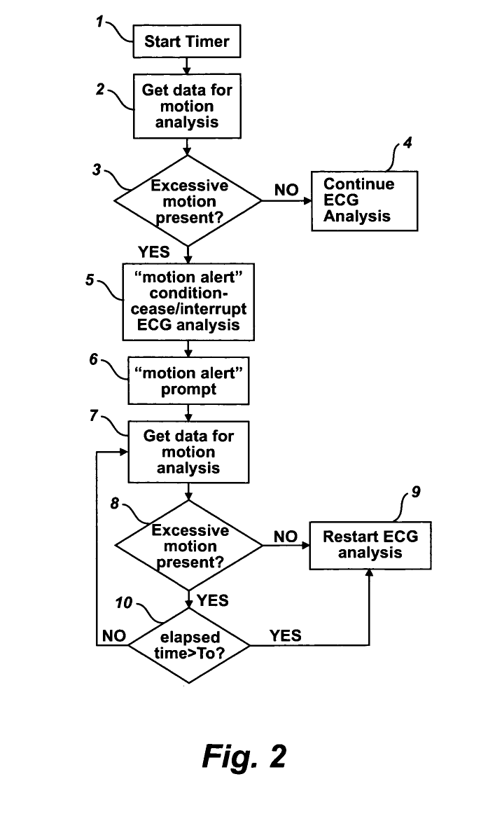

Referring to FIG. 2, a process which may be performed by the processor 26 is shown. In the illustrated embodiment, the AED will perform analysis of ECG data such as an ECG analysis to see if the patient has a heart rhythm that calls for defibrillation therapy (such as, for example, the Shock Advisory System™ analysis used in the LIFEPAK CR Plus and LIFEPAK 500 defibrillators), or any other ECG analysis (such as, for example, a VF waveform analysis which evaluates the likelihood that a defibrillation shock will be effective), or a combination of such ECG analyses.

In the illustrated embodiment, when an ECG analysis starts, an internal timer is also started (block 1). Concurrent with the ECG analysis, data is collected for motion analysis (block 2). This data may be patient impedance, and motion detection may be determined by data analysis such as that discussed above, to determine if excessive motion is present (block 3). If no excessive motion is determined to be present, the device continues the ongoing ECG analysis (block 4). If excessive motion is determined to be present, and the internal timer has not yet exceeded a predetermined elapsed time T₀, the device enters into a “motion alert” condition and interrupts or ceases the ECG analysis (block 5). The device then prompts the rescuer to stop motion (block 6). For example, it may give an audible voiced prompt such as “Motion detected, stop motion”, or “Motion detected, do not touch patient”, or “Analyzing interrupted, stop all motion.” If the rescuer had been performing CPR, this prompt will alert him to take his hands off the patient. Next, data for motion analysis continues to be collected (block 7), and a determination of the presence or absence of excessive motion is made (block 8). If excessive motion is no longer present, then the device continues on to the step of block 9, where the interrupted ECG analysis is restarted. The ECG analysis may be started again from the beginning, or it may be picked up where it had left off at block 5. Once the internal timer has exceeded a predetermined elapsed time T₀, the device will start or continue the ECG analysis regardless of the presence of motion.

The time period T₀ is preferably short enough to minimize the delay in therapy (for example, less than 30 seconds), and long enough that the user has an opportunity to address any motion conditions that are easily addressable (for example, 5 seconds to stop CPR). A value for T₀ such as 10 seconds may be appropriate.

The time period T₀ may be preset. Alternatively, it may be configured at a desired value upon initial setup of the defibrillator device, or in other embodiments, it may be configurable as desired (via the user interface, for example or via a reprogramming of the defibrillator) at any point in the operational life of the defibrillator.

The defibrillator may be equipped with a user input mechanism so that, if a user wants to override the automatic restart of the ECG analysis after the pre-programmed delay, the defibrillator may be instructed to not continue ECG analysis after a time T₀ but instead to wait, either for an extended time period of a preset length, or until further input from the user is received by the defibrillator. This may be useful in cases where, for example, a user is making a deliberate choice to cause motion sufficient to interfere with accurate ECG analysis (for example, where a user is moving a patient to another location, or choosing to administer a therapy that causes motion). The user input may be accomplished through, for example, a user interface that includes a button to be pressed to indicate the override choice.

The process and steps shown in FIG. 2 are for illustrative purposes only. Alternative embodiments are possible. For example, instead of starting a timer in the step of block 1 to track when the time interval T₀ has been reached, the processor could note the time of step block 5 on an internal clock, calculate elapsed time in block 10 and compare it to the desired threshold value for elapsed time.

Using a process such as that described above or an equivalent after excessive motion is first detected, ECG analysis will begin to proceed again when the motion detection condition is cleared or at the end of the aforementioned period of time T₀, whichever occurs first. The pause in ECG analysis resulting from the motion detection condition will not last longer than the aforementioned period of time. This will provide a defibrillator which prompts the user to halt actions that may result in ECG artifact, and which can be quickly addressed, such as halting chest compressions. Likewise, motion due to agonal breathing, electromagnetic interference, or some other cause beyond the control of the rescuers, and which may not necessarily result in ECG artifact, will not result in excessively long or indefinite delay in ECG analysis. In the event of continuous or intermittent motion detection, ECG analysis will proceed after the aforementioned period has transpired regardless of whether or not motion detection persists; ECG analysis will not be delayed indefinitely. The completion of ECG analysis will allow the rescuer to proceed with either CPR or a defibrillation shock, both of which may be immediately beneficial to the patient in many circumstances.

In the rare event that continuous motion detection is caused by electromagnetic interference, the described embodiment of a motion detection system will allow ECG analysis to proceed after the short period of delay.

It will be apparent to those skilled in the art that various modifications and variations can be made to the above-described embodiment(s) of the invention without departing from the spirit and scope of the invention. Thus, it is intended that the present invention cover the modifications and variations of the embodiments provided they come within the scope of the appended claims and their equivalents.

What is claimed is:

1. A method of operating a medical device capable of analyzing a patient's ECG comprising the steps of:
(a) determining if the patient is undergoing motion;
(b) if it is determined that the patient is undergoing motion, providing an indication of patient motion;
(c) after providing the indication, determining if the patient is undergoing motion, and if the patient is undergoing motion, starting ECG analysis after an elapsed time T₀.

2. The method of claim 1 wherein the elapsed time T₀ is measured from a point in time prior to the commencement of step (a).

3. The method of claim 1 further comprising the steps of collecting ECG data of the patient, and initiating analysis of the ECG data prior to step (b).

4. The method of claim 3 wherein the step (b) further includes the step of interrupting the prior initiated ECG analysis.

5. The method of claim 1 wherein T₀ is substantially equal to or less than 30 seconds.

6. The method of claim 5 wherein T₀ is substantially equal to or less than 10 seconds.

7. The method of claim 3 wherein determining if the patient is undergoing motion includes sensing patient impedance and analyzing the sensed patient impedance.

8. The method of claim 4 wherein the step of starting the ECG analysis after an elapsed time T₀ includes the step of continuing the ECG analysis that had been interrupted in step (b).

9. The method of claim 4 wherein the step of starting the ECG analysis after an elapsed time T₀ includes the step of commencing a new ECG analysis.

10. The method of claim 1 wherein the indication includes a prompt informing the user that motion is present.

11. The method of claim 10 wherein the prompt includes an instruction to the user to stop motion.

12. The method of claim 10 wherein the prompt includes an instruction to the user to not touch the patient.

13. The method of claim 3 further comprising the step of starting a timer when the ECG analysis is commenced to measure elapsed time.

14. A defibrillator comprising:
electrodes attachable to a patient and configured to sense ECG signals and impedance; and
a processor capable of analyzing the sensed impedance to detect if motion is present; and, if motion is present, waiting for an elapsed time T₀ before analyzing the ECG signals.

15. The defibrillator of claim 14 further comprising a user interface controlled by the processor, and wherein the processor causes the user interface to inform the user if the processor determines that motion is present.

16. The defibrillator of claim 15 wherein the user interface provides a prompt to the user to stop motion if the processor determines that motion is present.

17. The defibrillator of claim 16 wherein the prompt includes a voice prompt instructing the user to not touch the patient.

18. A method of operating a defibrillator having electrodes attached to a patient comprising the steps of:
starting an ECG analysis of the patient;
starting a measurement of elapsed time when the ECG analysis is started;
analyzing a patient parameter to determine if the patient is undergoing motion which would influence ECG analysis;
if the analyzing step determines that the patient is undergoing motion which would influence ECG analysis, and the time measurement shows that an elapsed time of T₀ has not been reaches, interrupting the ECG analysis.

19. The method of claim 18 further comprising the step of: after a time T₀ has been reached, analyzing the ECG.

20. The method of claim 19 wherein the step of analyzing the ECG after a time T₀ has been reached includes the step of continuing the ECG analysis which had been interrupted.

21. The method of claim 19 wherein the step of analyzing the ECG after a time T₀ has been reached includes the step of commencing a new ECG analysis.

22. The method of claim 18 further comprising the step of
if the analyzing step determines that the patient is undergoing motion which would influence ECG analysis and the time measurement shows that an elapsed time of T₀ has not been reached, analyzing a second data set of a patient parameter to determine if the patient is undergoing motion which would influence ECG analysis; and if motion which would influence ECG analysis is present, waiting for an elapsed time T₀ before analyzing the ECG signals.

23. The method of claim 18 wherein the patient parameter is impedance.

24. The method of claim 18 wherein T₀ is substantially equal to 30 seconds.

25. The method of claim 18 wherein T₀ is substantially equal to 10 seconds.

26. The method of claim 18 wherein the time T₀ is a time that is may be chosen prior to application of the electrodes to the patient.
